(19) United States
(12) Patent Application Publication
(10) Pub. No.: US 2013/0122606 A1
(43) Pub. Date: May 16, 2013

(54) METHOD AND DEVICE FOR CHEMICAL AND/OR BIOLOGICAL ANALYSIS

(71) Applicants: Elisabeth Cerrato, Lyon (FR); Sylvie Cheucle, La Tour de Salvagny (FR); Bruno Colin, Marcy L'Etoile (FR); Virginie Helleux, Lyon (FR); Cecile Paris, Bessenay (FR)

(72) Inventors: Elisabeth Cerrato, Lyon (FR); Sylvie Cheucle, La Tour de Salvagny (FR); Bruno Colin, Marcy L'Etoile (FR); Virginie Helleux, Lyon (FR); Cecile Paris, Bessenay (FR)

(73) Assignee: BIOMERIEUX, MARCY L'ETOILE (FR)

(21) Appl. No.: 13/810,998

(22) PCT Filed: Jul. 28, 2011
(86) PCT No.: PCT/FR11/51819

§ 371 Date: Jan. 18, 2013

(30) Foreign Application Priority Data
Aug. 3, 2010 (FR) 1056401

(51) Int. Cl. G01N 33/53

(52) U.S. Cl. CPC G01N 33/5306
USPC 436/501; 422/69

(57) ABSTRACT

A method of chemical and/or biological analysis including: providing a container delimited by a wall including an inner surface on which there is deposited an active component intended to make possible a chemical and/or biological reaction with a solution disposed in the container; filling the container with a liquid solution containing an analyte which is to be detected; and in applying to the liquid a succession of movements of aspirating/discharging a volume in the container at a suitable frequency to bring about resonance of the liquid so as to deform the free surface thereof and to result in a preferential distribution of the fluid along the inner surface of the wall of the container.

The present invention relates to automated apparatuses for performing chemical or biological tests, and in particular immunological tests.

Medical biology plays a major role in public health, whether in terms of diagnosis of the illness, patient care and his/her treatment. Automated instruments intended for medical analyses are therefore commonly used in laboratories. There are numerous automated diagnostic apparatuses on the market which particularly make it possible, by immunological assays, such as an ELISA test, to isolate pathogens responsible for numerous illnesses. These immunological analysis machines generally employ successive automated steps of injection/aspiration of the biological sample which is desired to be analysed and of reagents for the detection of one or more given pathogens. For example, these machines make it possible to quickly develop an ELISA (Enzyme Linked ImmunoSorbent Assay) type sandwich test for a large number of samples.

Thus, as an indication, the first step can consist of depositing on the walls of a solid support an active component, particularly in the form of a capture antibody, specific to one or more target antigens of a given pathogen which is desired to be isolated in a biological sample of a patient. The second step then consists of placing the biological sample of the patient in contact with said capture antibodies. If the sample of the patient contains the target antigen, a complex forms between the target antigen and the capture antibody. The target antigens fixed on the capture antibodies are then placed in contact with detection antibodies, which are marked, in order to reveal the presence of the capture antibody—target antigen—detection antibody sandwich. Such tests are well known to the person skilled in the art.

As an example of automated apparatuses for immunological assays, we can mention in particular the VIDAS® apparatus marketed by the applicant which is a multiparameter machine for immunoanalyses. This apparatus is composed of an analytical module which automatically deals with all of the analysis steps until results are yielded. The principle of the VIDAS® test is based on the use of a cone and a reaction well strip.

The cone constitutes the solid phase of the reaction on the wall of which the capture antibodies are fixed. It is single-use and is used for sampling. It makes possible, during the test, the steps of aspirating/discharging intended to successively place in contact the multiple reagents involved in the test.

The well strip contains all of the reagents ready for use, distributed into the different wells of the well strip, and in particular the detection antibodies. A well is also for the depositing of the sample which is desired to be analysed. At each immunoanalysis step, the cone aspirates and discharges, several times, the different reagents contained in the wells of the well strip up to the final reading step. The last well of the well strip constitutes the reading chamber in which the intensity of the reaction is measured in fluorescence.

This type of device is satisfactory in that it makes it possible to perform an efficient analysis. It is however desirable to enable faster execution of the tests to make it possible to provide the analysis results more rapidly.

To this end, the object of the present invention is a method of chemical and/or biological analysis comprising the steps consisting in:

providing a container delimited by a wall comprising an inner surface on which there is fixed an active component intended to make possible a chemical and/or biological reaction with a solution disposed in the container;

filling the container with a liquid solution containing an analyte which is to be detected;

applying to the liquid a succession of movements of aspirating/discharging a volume in the container at a suitable frequency to bring about resonance of the liquid so as to deform the free surface thereof and to result in a preferential distribution of the fluid along the inner surface of the wall of the container.

Due to the arrangements according to the invention, it appears that the contact surface between the active inner surface of the container onto which the active component is fixed and the solution contained in the container is significantly increased, and that the reaction between the analyte to be detected in the solution and the active component fixed onto the inner surface of the wall of the container is thus made easier which makes it possible to obtain an analysis result more quickly.

Advantageously, the frequency of the aspirating/discharging movements is between 0.2 and 20 Hz, preferably between 1 and 15 Hz, and in particular between 2 and 10 Hz.

Advantageously, the volume aspirated and discharged during the succession of aspirating and discharging movements in the container is between 0.5 and 360 μL, preferably between 1 and 50 μL.

Another object of the present invention is a device intended for the implementation of a method such as described above, comprising:

at least one container delimited by a wall comprising an inner surface onto which there is fixed an active component intended to make possible a chemical and/or biological reaction with a solution disposed in the container;

means for aspirating/discharging a liquid disposed in the container;

the aspirating/discharging means being arranged to perform aspirating/discharging movements in the container at a suitable frequency to bring about a resonance of the liquid so as to deform the free surface thereof and to result in a preferential distribution of the fluid along the inner surface of the wall of the container.

Advantageously, the container is conical in shape.

According to an embodiment, the device further includes a well intended for receiving a liquid solution, and means for relative displacement of said well relative to the container, the displacement means being arranged to displace the container between a first position, the low position, in which an orifice of the container is in contact with the liquid solution so as to allow the aspiration of said solution from the well in the container, and a second position, the high position, in which the orifice of the container is disposed above the free surface of the liquid solution present in the well, in which position the succession of aspirating/discharging movements is applied to the solution present in the container.

In the following detailed description of the figures defined below, the same elements or elements fulfilling identical functions may retain the same references in order to make the invention easier to understand.

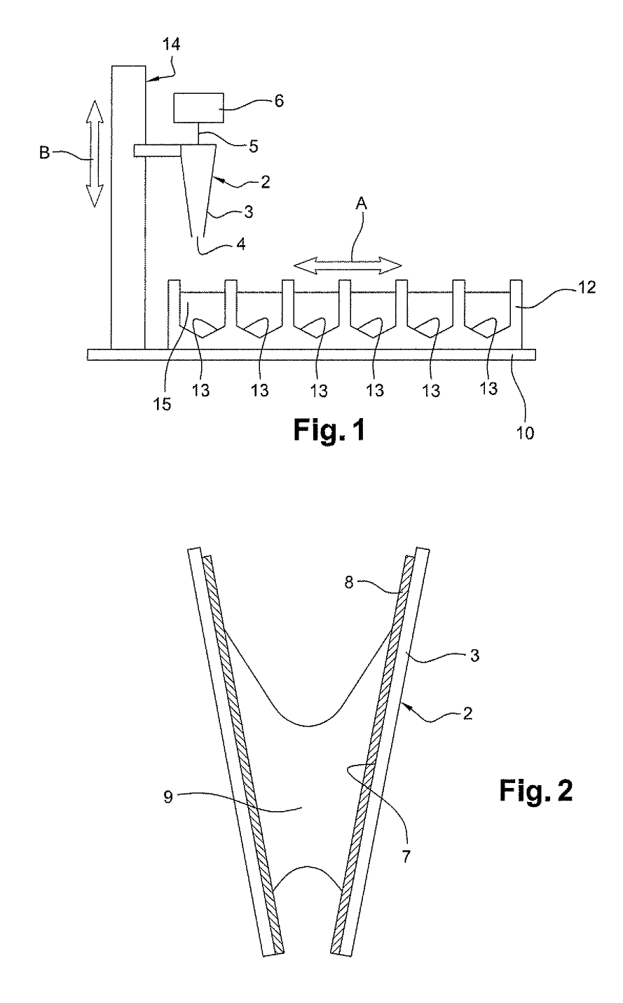

FIG. 1 is a schematic representation of a device according to the invention.

FIG. 2 schematically depicts the distribution of a liquid during aspirating/discharging operations in a container of the device from FIG. 1 during the application of a method according to the invention.

According to an embodiment depicted in FIG. 1, a device according to the invention comprises a cone-shaped container 2 delimited by a wall 3 which has an opening 4 at its point to allow the aspiration or the discharging of liquid.

The upper part of the cone is linked by fluid communication means 5 to means 6 for aspirating/discharging a liquid in the cone 2.

The inner surface 7 of the wall of the cone comprises a deposit 8 of an active component intended to make possible a chemical and/or biological reaction with a solution 9 disposed in the cone 2.

The device also comprises means 10 for receiving a reaction well strip 12 containing reagents ready for use, distributed in the different wells 13 of the well strip. By way of example, the reagents can be constituted by detection antibodies. According to a known arrangement, one of the wells 13 can also be for the depositing of the sample which is desired to be analysed.

The device furthermore comprises means 14 for the support and relative displacement of the cone relative to the reaction well strip containing the wells. The displacement means are arranged to make it possible to move along the axis of the well strip as indicated by the arrow A in order to position the point of the cone 3 facing a particular well, and a movement perpendicular to the axis of the well strip indicated by the arrow B, in order to allow the end 4 of the cone 2 to penetrate into the well 13 to sample a solution present in the well or to pour the content of the cone 2 into said well.

It is known to use the aspirating/discharging means 6 to allow the aspiration and the discharging of the liquid between a well 13 and the cone 2 or reaction container.

Thus due to the displacement means 14, the cone 2 is displaced facing a well 13, then the end 4 of the cone 2 is introduced into the well until it penetrates into a solution 15 contained in the well 13.

An aspiration of a determined quantity of solution is performed, using the aspiration means 6.

The cone is then displaced by the displacement means 14 to reach a high position or mixing position, in which the end of the cone 2 is above the free surface of the solution present in the well 13.

It can be envisaged that the end of the cone 2 be placed above the surface of solution present in the well, but remaining within the enclosure of the well.

According to the arrangements according to the invention, in the mixture position defined above, the aspiration means 6 are arranged to generate aspirating/discharging movements in the cone 2 at a suitable frequency to bring about resonance of the liquid 9 so as to deform the free surface thereof and to result in a distribution of the fluid along the inner surface 7 of the wall of the cone 2.

The preferential distribution of the liquid in the cone is in that case realised as depicted in FIG. 2, with a larger quantity of liquid being present on the wall of the container compared to the quantity disposed in the centre of the container.

Due to these arrangements, the contact surface between the liquid contained in the cone and the inner surface 7 of the wall of the cone is increased, and thus the reaction between the elements to be detected in the solution and the active component forming a deposit 8 on the wall is accelerated.

The frequency f of the aspirating/discharging movements which makes it possible to obtain the phenomenon of resonance and preferential distribution of the liquid along the walls of the cone is between 0.2 and 20 Hz, preferably between 1 and 15 Hz, and preferably between 2 and 10 Hz,

The quantity of volume v aspirated and discharged in the container is between 0.5 and 360 μL.

According to an embodiment, for a volume of 20 μL contained in the cone, an amplitude at volume v of the aspirating/discharging movements of around 5 μL can be advantageously used, and a discharge frequency of between 4.3 and 4.5 Hz.

Although the invention has been described in connection with particular embodiments, it is evident that it is by no means limited thereby and that it comprises all of the technical equivalents of the means described and combinations thereof if they fall within the scope of the invention.

1. A method of chemical and/or biological analysis comprising the steps of:
providing a container delimited by a wall comprising an inner surface on which there is fixed an active component intended to make possible a chemical and/or biological reaction with a solution disposed in the container;
filling the container with a liquid solution containing an analyte which is to be detected;
applying to the liquid a succession of movements of aspirating/discharging a volume in the container at a suitable frequency to bring about resonance of the liquid so as to deform the free surface thereof and to result in a preferential distribution of the fluid along the inner surface of the wall of the container.

2. The method according to claim 1, wherein the frequency of the aspirating/discharging movements is between 0.2 and 20 Hz.

3. The method according to claim 1, wherein the frequency of the aspirating/discharging movements is between 1 and 15 Hz.

4. The method according to claim 1, wherein the volume aspirated and discharged during the succession of aspirating and discharging movements in the container is between 0.5 and 360 μL.

5. The method according to claim 1, wherein the volume aspirated and discharged during the succession of aspirating and discharging movements in the container is between 1 and 50 μL.

6. A device intended for the implementation of a method according to claim 1, wherein the device comprises:
at least one container delimited by a wall comprising an inner surface onto which there is fixed an active component intended to make possible a chemical and/or biological reaction with a solution disposed in the container;
means for aspirating/discharging a liquid disposed in the container;
the aspirating/discharging means being arranged to perform aspirating/discharging movements in the container at a suitable frequency to bring about resonance of the liquid so as to deform the free surface thereof and to result in a preferential distribution of the fluid along the inner surface of the wall of the container.

7. The device according to claim 6, wherein the container is conical in shape.

8. The device according to claim 6, wherein the device further includes a well intended for receiving a liquid solution, and means for relative displacement of said well relative to the container, the displacement means being arranged to displace the container between a first position, the low position, in which an orifice of the container is in contact with the liquid solution so as to allow the aspiration of said solution from the well in the container, and a second position, the high position, in which the orifice of the container is disposed above the free surface of the liquid solution present in the well, in which position the succession of aspirating/discharging movements is applied to the solution present in the container.
